(19) United States
(12) Patent Application Publication
(10) Pub. No.: US 2018/0036100 A1
(43) Pub. Date: Feb. 8, 2018

(54) METHOD AND SYSTEM FOR PROVIDING 3D TEETH ALIGNMENT IMAGE DISPLAY SERVICES, AND COMPUTER-READABLE MEDIUM ON WHICH THE METHOD IS RECORDED

(71) Applicant: eClear International Co., Ltd., Los Angeles, CA (US)

(72) Inventor: TAEWEON KIM, Seoul (KR)

(21) Appl. No.: 15/228,043

(22) Filed: Aug. 4, 2016

(51) Int. Cl. A61C 7/00; A61B 5/00; G03H 1/22; G06T 19/20; G03H 1/00; G03H 1/08; A61B 1/24; A61C 9/00

(52) U.S. Cl. CPC A61C 7/002; A61B 1/24; A61B 5/0077; A61B 5/4803; A61C 9/004; A61B 5/0013; A61B 5/745; G06T 19/20; G03H 1/0005; G03H 1/08; G03H 1/2205; A61B 2090/367

(57) ABSTRACT

In a method of providing 3D teeth alignment image display services using a computer system including at least one orthodontist terminal and a server computer connected to a network, a target teeth information on an orthodontic patient may be received from the orthodontist terminal or an orthodontic patient terminal such that three or four images of a display are respectively projected onto a pyramidal three-sided or four-sided projection plate and thus the orthodontic patient checks a 3D hologram of his/her virtually aligned teeth, a virtually aligned teeth information that is obtained based on the target teeth information may be transformed to three-view or four-view image information outputtable by the display such that the virtually aligned teeth information is projectable onto the three-sided or four-sided projection plate, and the three-view or four-view image information may be transmitted to the orthodontist terminal.

### BACKGROUND

### 1. Field

The present invention relates to a method and system for providing 3D teeth alignment image display services, and a computer-readable medium on which the method is recorded and, more particularly, to a method and system for providing 3D teeth alignment image display services to display virtual teeth alignment images as a 3D hologram image to an orthodontic patient, and a computer-readable medium on which the method is recorded.

### 2. Description of the Related Art

Orthodontic treatment may be classified into metal-brace treatment using metal braces, lingual treatment using lingual braces, and clear-aligner treatment using clear aligners.

In particular, clear-aligner treatment attracts people's attention compared to the other orthodontic treatment methods because a transparent clear aligner covering teeth like a mouth piece is invisible, detachable, and easily wearable.

The Internet consists of a huge number of computers and computer networks for globally interconnecting the computers using communication links.

The interconnected computers exchange information using a variety of services, e.g., e-mails, Gopher, and World Wide Web (WWW). The World Wide Web allows a server computer system (e.g., a web server or a website) to transmit graphical webpages of information to a remote client computer system.

The remote client computer system may display various webpages received from the server computer.

Due to rapid development of wireless communication networks including a mobile communication network such as a second generation (2G), third generation (3G), fourth generation (4G), fifth generation (5G), or long term evolution (LTE) network, a WIFI communication network, a Bluetooth communication network, a cellular communication network, a code division multiple access (CDMA) communication network, an Ethernet communication network, a WiMAX communication network, a local area network (LAN), a wide area network (WAN), a radio frequency (RF) communication network, an infrared communication network, an optical communication network, and a near field communication (NFC) network, a variety of systems are being developed using the wireless communication networks.

According to the conventional orthodontic treatment methods using orthodontic appliances such as clear aligners, since orthodontic patients are able to actually check their aligned teeth or face contours only after orthodontic treatment is finished and are not able to accurately know the same before orthodontic treatment is finished, reliability of orthodontic treatment may not be high. As such, many people may decide not to receive orthodontic treatment and thus may not have their teeth aligned.

In addition, orthodontic patients should imagine their aligned teeth or face contours using only 2D images which are not appropriate to express lifelike teeth or face contours of people, and the inappropriate 2D images increase dissatisfaction of the orthodontic patients and thus are unhelpful for business, advertisement, or promotion.

### SUMMARY

The present invention provides a method and system for providing 3D teeth alignment image display services by inputting a target teeth information on an orthodontic patient from an orthodontist to a server computer, transforming the target teeth information into a virtually aligned teeth information, transforming the virtually aligned teeth information into three-view or four-view image information, transmitting the three-view or four-view image information to the orthodontist or an orthodontic appliance manufacturer, and displaying a 3D teeth alignment image using a 3D holographic device of the orthodontist, to increase reliability of orthodontic treatment, to increase confidence and satisfaction of the orthodontic patient in orthodontic treatment, to achieve advantages in business, advertisement, or promotion, and to provide differentiated and high-value-added services, and a computer-readable medium having recorded the method thereon. However, the scope of the present invention is not limited thereto.

According to an aspect of the present invention, there is provided a method of providing 3D teeth alignment image display services using a computer system including at least one orthodontist terminal and a server computer connected to a network, by receiving a target teeth information of an orthodontic patient from the orthodontist terminal or an orthodontic patient terminal such that three or four images of a display are respectively projected onto a pyramidal three-sided or four-sided projection plate and thus the orthodontic patient checks a 3D hologram of his/her virtually aligned teeth, transforming a virtually aligned teeth information that is obtained based on the target teeth information, into three-view or four-view image information outputtable by the display such that the virtually aligned teeth information is projectable onto the three-sided or four-sided projection plate, and transmitting the three-view or four-view image information to the orthodontist terminal.

According to another aspect of the present invention, there is provided a method of providing 3D teeth alignment image display services using a computer system including at least one orthodontist terminal and a server computer connected to a network, the server computer including a target teeth information input program for receiving a target teeth information on an orthodontic patient from the orthodontist terminal or an orthodontic patient terminal such that three or four images of a display are respectively projected onto a pyramidal three-sided or four-sided projection plate and thus the orthodontic patient checks a 3D hologram of his/her virtually aligned teeth, a three-view or four-view image information transformation program for transforming a virtually aligned teeth information that is obtained based on the target teeth information, into three-view or four-view image information outputtable by the display such that the virtually aligned teeth information is projectable onto the three-sided or four-sided projection plate, a three-view or four-view image information transmission program for transmitting the three-view or four-view image information to the orthodontist terminal, a target teeth information database for storing the target teeth information, and a three-view or four-view image information database for storing the three-view or four-view image information, and the method including (a) receiving the target teeth information on the orthodontic patient from the orthodontist terminal or the orthodontic patient terminal using the target teeth information input program, (b) transforming the virtually aligned teeth information that is obtained based on the target teeth information, to the three-view or four-view image information outputtable by the display such that the virtually aligned teeth information is projectable onto the three-sided or four-sided projection plate, using the three-view or four-view image information transformation program, and (c) transmitting the three-view or four-view image information to the orthodontist terminal using the three-view or four-view image information transmission program.

The target teeth information may include at least one of a teeth photo information obtained by photographing teeth or dental impressions of the orthodontic patient; a 3D-scanned teeth information obtained by 3D-scanning the teeth or the dental impressions of the orthodontic patient; a face photo information obtained by photographing a face of the orthodontic patient; a 3D-scanned face information obtained by 3D-scanning the face of orthodontic patient; a current mouth shape information of the orthodontic patient; a current voice information of the orthodontic patient; a current facial expression information of the orthodontic patient and combinations thereof.

The three-view or four-view image information may include at least one of a front view image information, a left side view image information, a right side view image information, and a rear view image information and combinations thereof, indicating virtually aligned teeth or face contours of the orthodontic patient, and the front view image information, the left side view image information, the right side view image information, and the rear view image information may be information on images which are radially provided in three or four equiangular directions about a point that corresponds to an apex of the three-sided or four-sided projection plate and thus are respectively projectable onto each side of the three-sided or four-sided projection plate.

The server computer may further include a virtual alignment program for virtually aligning the target teeth information into the virtually aligned teeth information, and a virtually aligned teeth information database for storing the virtually aligned teeth information, and the method may further include (d) virtually aligning the target teeth information into the virtually aligned teeth information using the virtual alignment program, after step (a).

The virtual alignment program may be a program for virtually aligning the target teeth information into the virtually aligned teeth information by virtually moving anterior teeth in the target teeth information such that a front line of the anterior teeth meets an aesthetic guide line corresponding to a virtual line for connecting a first skin point located in front of a nasion from which nasal bones start based on a profile skull line of a person, to a second skin point located in front of a pogonion, and then moving other teeth onto virtual and ideal correction arcs based on the moved anterior teeth.

The virtually aligned teeth information may include at least one of a virtually corrected teeth information of the orthodontic patient; a virtually corrected face contour information of the orthodontic patient; a virtually corrected mouth shape information of the orthodontic patient; a virtually corrected voice information of the orthodontic patient; a virtually corrected facial expression information of the orthodontic patient; and combinations thereof.

The server computer may further include a comparison program for receiving a completely aligned teeth information on completely aligned teeth of the orthodontic patient from the orthodontist terminal or the orthodontic patient terminal, transforming an actually aligned teeth information to three-view or four-view image information outputtable by the display such that the actually aligned teeth information is projectable onto the three-sided or four-sided projection plate to be compared with the virtually aligned teeth information, and transmitting the three-view or four-view image information to the orthodontist terminal; and an actually aligned teeth information database for storing the actually aligned teeth information, and the method may further include (e) receiving the completely aligned teeth information on the completely aligned teeth of the orthodontic patient from the orthodontist terminal or the orthodontic patient terminal, transforming the actually aligned teeth information to the three-view or four-view image information outputtable by the display such that the actually aligned teeth information is projectable onto the three-sided or four-sided projection plate to be compared with the virtually aligned teeth information, and transmitting the three-view or four-view image information to the orthodontist terminal, using the comparison program, after step (c).

According to another aspect of the present invention, there is provided a computer system for providing 3D teeth alignment image display services using a computer system comprising at least one orthodontist terminal and a server computer connected to a network, the server computer including a target teeth information input program for receiving a target teeth information on an orthodontic patient from the orthodontist terminal or an orthodontic patient terminal such that three or four images from a display are respectively projected onto a substantially pyramidal three-sided or four-sided projection plate and thus the orthodontic patient checks a 3D hologram of his/her virtually aligned teeth; a three-view or four-view image information transformation program for transforming a virtually aligned teeth information that is obtained based on the target teeth information, to three-view or four-view image information outputtable by the display such that the virtually aligned teeth information is projectable onto the three-sided or four-sided projection plate; a three-view or four-view image information transmission program for transmitting the three-view or four-view image information to the orthodontist terminal; a target teeth information database for storing the target teeth information; a three-view or four-view image information database for storing the three-view or four-view image information; and a controller programmed to receive the target teeth information on the orthodontic patient from the orthodontist terminal or the orthodontic patient terminal using the target teeth information input program, to transform the virtually aligned teeth information that is obtained based on the target teeth information, to the three-view or four-view image information outputtable by the display such that the virtually aligned teeth information is projectable onto the three-sided or four-sided projection plate, using the three-view or four-view image information transformation program, and to transmit the three-view or four-view image information to the orthodontist terminal using the three-view or four-view image information transmission program.

According to another aspect of the present invention, there is provided a computer-readable medium on which is recorded a method of providing 3D teeth alignment image display services using a computer system comprising at least one orthodontist terminal and a server computer connected to a network, the server computer including a target teeth information input program for receiving a target teeth information on an orthodontic patient from the orthodontist terminal or an orthodontic patient terminal such that three or four images from a display are respectively projected onto a pyramidal three-sided or four-sided projection plate and thus the orthodontic patient checks a 3D hologram of his/her virtually aligned teeth; a three-view or four-view image information transformation program for transforming a virtually aligned teeth information that is obtained based on the target teeth information, to three-view or four-view image information outputtable by the display such that the virtually aligned teeth information is projectable onto the three-sided or four-sided projection plate; a three-view or four-view image information transmission program for transmitting the three-view or four-view image information to the orthodontist terminal; a target teeth information database for storing the target teeth information; and a three-view or four-view image information database for storing the three-view or four-view image information, and the method including (a) receiving the target teeth information on the orthodontic patient from the orthodontist terminal or the orthodontic patient terminal using the target teeth information input program; (b) transforming the virtually aligned teeth information that is obtained based on the target teeth information, to the three-view or four-view image information outputtable by the display such that the virtually aligned teeth information is projectable onto the three-sided or four-sided projection plate, using the three-view or four-view image information transformation program; and (c) transmitting the three-view or four-view image information to the orthodontist terminal using the three-view or four-view image information transmission program.

### BRIEF DESCRIPTION OF THE DRAWINGS

The above and other features and advantages of the present invention will become more apparent by describing in detail embodiments thereof with reference to the attached drawings in which:

FIG. 1 is a conceptual view of a system for providing 3D teeth alignment image display services, according to some embodiments of the present invention;

FIG. 2 is a side view of a virtual face for describing a process for generating virtually aligned teeth information by a virtual alignment program of the 3D teeth alignment image display service providing system of FIG. 1;

FIG. 3 is a bottom view of virtual teeth for describing the process for generating the virtually aligned teeth information by the virtual alignment program of the 3D teeth alignment image display service providing system of FIG. 1;

FIG. 4 is a conceptual view showing the relationship among an operator for providing 3D teeth alignment image display services of the present invention, an orthodontic patient, an orthodontist, and an orthodontic appliance manufacturer;

FIG. 5 is a flowchart of a method of providing 3D teeth alignment image display services, according to some embodiments of the present invention;

FIG. 6 is a flowchart of a method of providing 3D teeth alignment image display services, according to other embodiments of the present invention; and

FIGS. 7 and 8 are bottom views showing examples of three-view or four-view image information transmittable to an orthodontist terminal and displayable by a display of a 3D holographic device using a 3D teeth alignment image display service providing system according to some embodiments of the present invention.

### DETAILED DESCRIPTION

Hereinafter, the present invention will be described in detail by explaining embodiments of the invention with reference to the attached drawings.

The invention may, however, be embodied in many different forms and should not be construed as being limited to the embodiments set forth herein; rather, these embodiments are provided so that this disclosure will be thorough and complete, and will fully convey the concept of the invention to one of ordinary skill in the art. In the drawings, the thicknesses or sizes of layers are exaggerated for clarity.

The terminology used herein is for the purpose of describing particular embodiments only and is not intended to be limiting of embodiments. As used herein, the singular forms “a,” “an” and “the” are intended to include the plural forms as well, unless the context clearly indicates otherwise. It will be further understood that the terms “comprises” and/or “comprising” when used in this specification, specify the presence of stated features, integers, steps, operations, elements, and/or components, but do not preclude the presence or addition of one or more other features, integers, steps, operations, elements, components, and/or groups thereof.

Embodiments of the invention are described herein with reference to schematic illustrations of idealized embodiments (and intermediate structures) of the invention. As such, variations from the shapes of the illustrations as a result, for example, of manufacturing techniques and/or tolerances, are to be expected. Thus, the embodiments of the invention should not be construed as limited to the particular shapes of regions illustrated herein, but are to include deviations in shapes that result, for example, from manufacturing.

A method and system for providing 3D teeth alignment image display services, and a computer-readable medium having recorded the method thereon, according to some embodiments of the present invention are now described in detail with reference to the attached drawings.

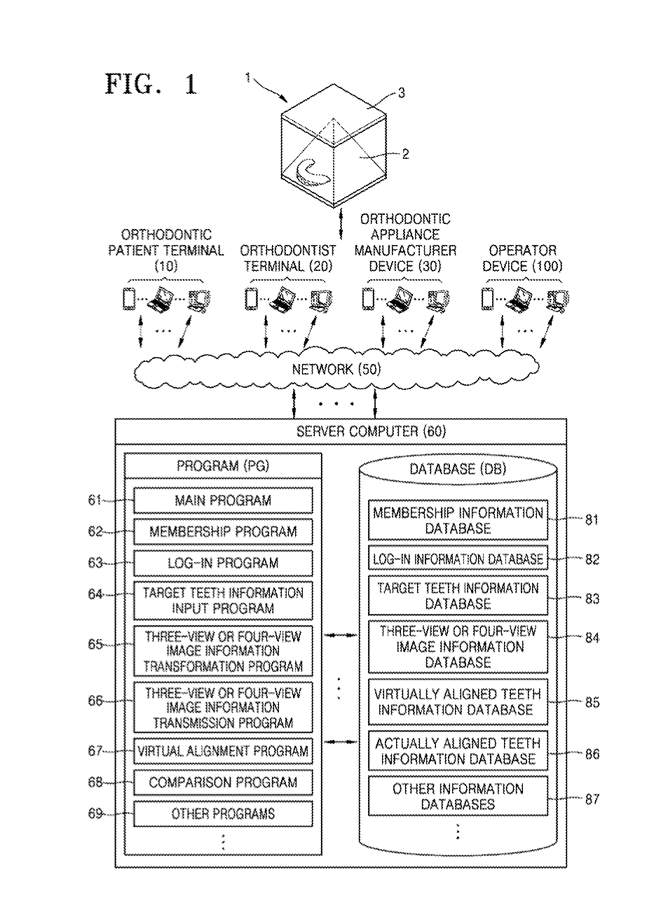

FIG. 1 is a conceptual view of a system for providing 3D teeth alignment image display services, according to some embodiments of the present invention.

Initially, as illustrated in FIG. 1, the 3D teeth alignment image display service providing system according to some embodiments of the present invention mainly includes an orthodontic patient terminal 10, an orthodontist terminal 20, an orthodontic appliance manufacturer device 30, an operator device 100, and a server computer 60 connected to the afore-mentioned devices through a network 50.

Herein, the server computer 60 is programmed to receive a target teeth information on the orthodontic patient from the orthodontist terminal 20 or the orthodontic patient terminal 10 such that three or four images of a display 3 are respectively projected onto a pyramidal three-sided or four-sided projection plate 2 of a 3D holographic device 1 and thus the orthodontic patient checks a 3D hologram of his/her virtually aligned teeth, to transform a virtually aligned teeth information that is obtained based on the target teeth information, to a three-view or four-view image information outputtable by the display 3 such that the virtually aligned teeth information is projectable onto the three-sided or four-sided projection plate 2, and to transmit the three-view or four-view image information to the orthodontist terminal 20, and may be a computer provided at a company, an agency, or a franchise capable of providing the 3D teeth alignment image display services through the network 50.

The server computer 60 is not limited to a server computer and may be one of a variety of computers or computing devices capable of functioning as servers.

For example, the orthodontic patient terminal 10 may be a device of the orthodontic patient who desires to check his/her virtually aligned teeth or face contour images as a 3D hologram, using the system of the present invention.

Herein, the orthodontic patient terminal 10 is not limited to a smartphone and may be one of all information devices capable of transmitting target teeth information. For example, the orthodontic patient terminal 10 may be a personal computer, a mobile device, a personal digital assistant (PDA), a smart pad, a laptop computer, a smart camera, a smart camcorder, an electronic book, a smart scanner, or the like.

For example, the orthodontist terminal 20 may be a device of the orthodontist, e.g., a dentist, a dental nurse, or a dental professional, who desires to provide differentiated and high-value-added services or to achieve advantages in business, advertisement, or promotion by providing aligned teeth images as a virtual 3D hologram image of the 3D holographic device 1 to the orthodontic patient, using the system of the present invention.

Herein, the orthodontist terminal 20 is not limited to a smartphone and may be one of a variety of cameras, 3D cameras, scanners, 3D scanners, etc. capable of inputting the target teeth information of the orthodontic patient, and all information devices capable of receiving or displaying image information used for a 3D hologram, e.g., the 3D holographic device 1 of FIG. 1. For example, the orthodontist terminal 20 may be a camera, a 3D camera, a scanner, a 3D scanner, a personal computer, a mobile device, a PDA, a smart pad, a laptop computer, a smart camera, a smart camcorder, an electronic book, a smart scanner, the 3D holographic device 1, or the like.

For example, the orthodontic appliance manufacturer device 30 may be a computer or a smartphone provided at a company, an agency, or a franchise which desires to manufacture a variety of orthodontic appliances, e.g., a clear aligner set and added or modified clear aligners to be provided to the orthodontic patient, using the system of the present invention.

Herein, the orthodontic appliance manufacturer device 30 is not limited to a smartphone and may be one of all information devices capable of receiving information about manufacturing of the orthodontic appliances. For example, the orthodontic appliance manufacturer device 30 may be a personal computer, a mobile device, a PDA, a smart pad, a laptop computer, a smart camera, a smart camcorder, an electronic book, a smart scanner, or the like.

For example, the operator device 100 is a device of a person, a company, an agency, or a franchise for managing and operating the server computer 60, is not limited to a computer or a smartphone, and may be an information device, a PDA, a smartphone, a smart pad, a camera, a camcorder, a laptop computer, an electronic book, a personal computer, or another server computer capable of receiving various types of text information, number information, or image information, and of selecting various commands.

Although the orthodontic appliance manufacturer device 30 and the operator device 100 are separately managed in FIG. 1, the present invention is not limited thereto. For example, the above-described operator device 100 may also serve as the orthodontic appliance manufacturer device 30. That is, the operator may also be the orthodontic appliance manufacturer. In addition, the operator may also be the orthodontist.

The orthodontic patient terminal 10, the orthodontist terminal 20, the orthodontic appliance manufacturer device 30, the operator device 100, and the server computer 60 of FIG. 1 are interconnected through the network 50 using various applications, hybrid applications, or programs installed thereon. The devices 10, 20, 30, and 40 interconnected through the network 50 may use a conventional mobile communication network such as a second generation (2G), third generation (3G), or fourth generation (4G) network, a WIFI communication network, a Bluetooth communication network, a cellular communication network, a code division multiple access (CDMA) communication network, a long term evolution (LTE) communication network, an Ethernet communication network, a WiMAX communication network, a local area network (LAN), a wide area network (WAN), a radio frequency (RF) communication network, an infrared communication network, an optical communication network, or a near field communication (NFC) network, and may have an Internet browser (e.g., Netscape or Internet Explorer) capable of displaying websites in the form of HTML, XML, or HTMLS, an internal, external, or short-distance/long-distance wired/wireless network access protocol device, or the like.

As illustrated in FIG. 1, the server computer 60 includes a program controller PG for controlling programs, and a database DB for storing various types of information.

Particularly, as illustrated in FIG. 1, the program controller PG may include a main program 61 for managing all the other programs, a membership program 62 for receiving a membership information from the orthodontic patient terminal 10, the orthodontist terminal 20, or the orthodontic appliance manufacturer device 30 and storing the membership information, a log-in program 63 for receiving a log-in information from the orthodontic patient terminal 10, the orthodontist terminal 20, or the orthodontic appliance manufacturer device 30, a target teeth information input program 64 for receiving the target teeth information on the orthodontic patient from the orthodontist terminal 20 or the orthodontic patient terminal 10 such that the three or four images of the display 3 are respectively projected onto the pyramidal three-sided or four-sided projection plate 2 and thus the orthodontic patient checks the 3D hologram of his/her virtually aligned teeth, a three-view or four-view image information transformation program 65 for transforming the virtually aligned teeth information that is obtained based on the target teeth information, to the three-view or four-view image information outputtable by the display 3 such that the virtually aligned teeth information is projectable onto the three-sided or four-sided projection plate 2, a three-view or four-view image information transmission program 66 for transmitting the three-view or four-view image information to the orthodontist terminal 20, a virtual alignment program 67 for virtually aligning the target teeth information into the virtually aligned teeth information, a comparison program 68 for receiving a completely aligned teeth information on completely aligned teeth of the orthodontic patient from the orthodontist terminal 20 or the orthodontic patient terminal 10, transforming an actually aligned teeth information to a three-view or four-view image information outputtable by the display 3 such that the actually aligned teeth information is projectable onto the three-sided or four-sided projection plate 2 to be compared with the virtually aligned teeth information, and transmitting the three-view or four-view image information to the orthodontist terminal 20, and other programs 69 for performing other functions, e.g., home page management, promotion, advertisement, data processing, cost management, and application installation.

Herein, the main program 61 manages all the other programs. The main program 61 may be implemented in the form of an on-line home page or a main page of an application, and may be a program for controlling all the other programs by receiving various command signals from the orthodontic patient terminal 10, the orthodontist terminal 20, the orthodontic appliance manufacturer device 30, and the operator device 100.

The membership program 62 may be a program for registering the orthodontic patient, the orthodontist, and the orthodontic appliance manufacturer as members. The membership program 62 may provide standard terms or terms for collecting and using personal information and may get an agreement thereof to receive the membership information from the orthodontic patient terminal 10, the orthodontist terminal 20, or the orthodontic appliance manufacturer device 30 and to store the membership information. For example, the membership information may include at least one of a real name, an Internet personal identification number (i-PIN), an identifier (ID), a password, an e-mail address, a mobile phone number, an address, personal information, a department name, a position name, an employee identification number, a resident registration number, and a unique number.

The log-in program 63 may be a program for receiving the log-in information, e.g., an ID or a password, required when the orthodontic patient, the orthodontist, or the orthodontic appliance manufacturer logs in the server computer 60, from the orthodontic patient terminal 10, the orthodontist terminal 20, or the orthodontic appliance manufacturer device 30.

The target teeth information input program 64 may be a program for receiving the target teeth information indicating a current state of teeth or face contours of the orthodontic patient from the orthodontist terminal 20 or the orthodontic patient terminal 10. For example, the target teeth information may include image or value information obtained from the orthodontic patient using a camera, a 3D camera, or a 3D scanner provided at a clinic of the orthodontist.

Accordingly, the orthodontist may transmit the target teeth information of the orthodontic patient, which is obtained by a camera or a scanner, through the network 50 to the server computer 60 using the target teeth information input program 64.

However, the person who inputs the target teeth information is not limited to the orthodontist, and the orthodontic patient may obtain the target teeth information by photographing or scanning himself or herself using the orthodontic patient terminal 10 and transmit the same through the network 50 to the server computer 60.

Alternatively, the person who inputs the target teeth information may be the orthodontic appliance manufacturer. In this case, the orthodontic appliance manufacturer may obtain the target teeth information by photographing or scanning the orthodontic patient using a camera, a 3D camera, or a 3D scanner provided at an office and transmit the same through the network 50 to the server computer 60.

Herein, for example, the target teeth information may include at least one of teeth photo information obtained by photographing teeth or dental impressions of the orthodontic patient, 3D-scanned teeth information obtained by 3D-scanning the teeth or the dental impressions of the orthodontic patient, face photo information obtained by photographing a face of the orthodontic patient, 3D-scanned face information obtained by 3D-scanning the face of orthodontic patient, current mouth shape information of the orthodontic patient, current voice information of the orthodontic patient, current facial expression information of the orthodontic patient, and combinations thereof.

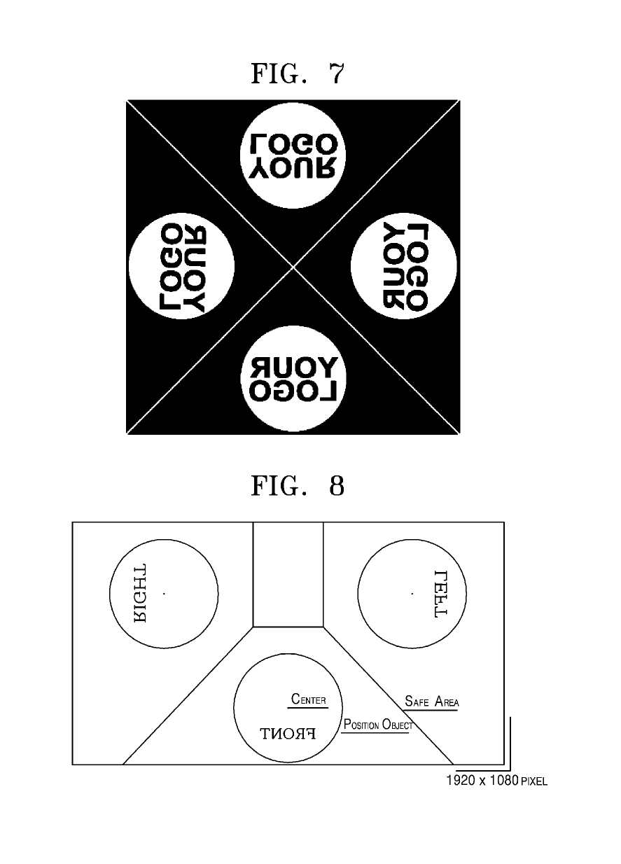

The three-view or four-view image information transformation program 65 may be a program for transforming the virtually aligned teeth information virtually aligned based on the target teeth information, into the three-view or four-view image information outputtable by the display 3 in such a manner that the virtually aligned teeth information is projectable onto the three-sided or four-sided projection plate 2. In detail, as illustrated in FIGS. 7 and 8 to be described below, the three-view or four-view image information transformation program 65 may be a program for providing different-view images of an object in such a manner that the display 3 simultaneously projects and displays the images as a 3D hologram in a 3D space.

Herein, for example, as illustrated in FIGS. 7 and 8, the three-view or four-view image information may be information that can be displayed by the display 3, and may include at least one of a front view image information, a left side view image information, a right side view image information, and a rear view image information, and combinations thereof, indicating virtually aligned teeth or face contours of the orthodontic patient. The front view image information, the left side view image information, the right side view image information, and the rear view image information may be information on images which are radially provided in three or four equiangular directions about a point that corresponds to an apex of the three-sided or four-sided projection plate 2 and thus are respectively projectable onto sides of the three-sided or four-sided projection plate 2.

However, the three-view or four-view image information may not always include the rear view image information, and the rear view image information may be omitted.

Herein, the positions, sizes, angles, distances, etc. of the image information included in the three-view or four-view image information may vary depending on the shape, size, and projection angle of the three-sided or four-sided projection plate 2, a viewer location, etc., and may be optimized to maximize the 3D hologram effect.

Accordingly, since the three-view or four-view image information is easily obtainable using the 3D teeth alignment image display service providing system of the present invention without using additional computing equipment and a technical professional, the orthodontist may provide high-value services to meet customer satisfaction and may achieve great advantages in business, promotion, or advertisement.

The three-view or four-view image information transmission program 66 may be a program for transmitting the three-view or four-view image information to the orthodontist terminal 20. The orthodontist terminal 20 may be one of a large variety of electronic devices.

That is, when the orthodontist terminal 20 is an information device such as a smartphone or a personal computer, the orthodontist may receive the three-view or four-view image information and output the same to the display 3 of the 3D holographic device 1 to display a 3D hologram image.

Alternatively, the orthodontist terminal 20 may also serve as the 3D holographic device 1. In this case, the orthodontist may output the three-view or four-view image information to the display 3 immediately after receiving the same, to display a 3D hologram image.

The virtual alignment program 67 may be a program for virtually aligning the target teeth information into the virtually aligned teeth information. The virtually aligned teeth information may be automatically generated using an automated internal algorithm. If necessary, partial correction may be manually performed.

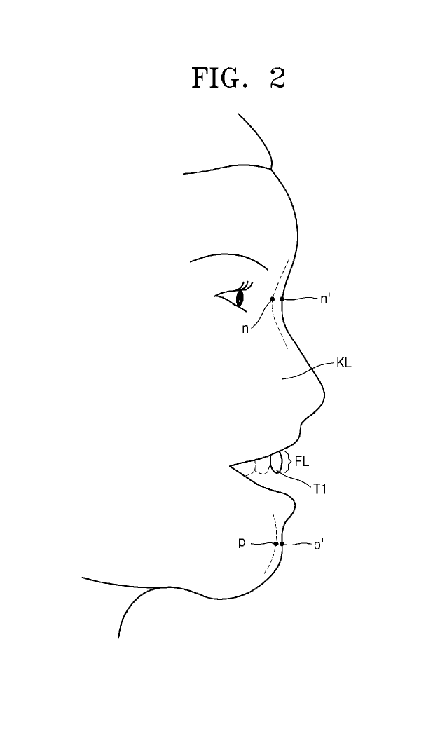

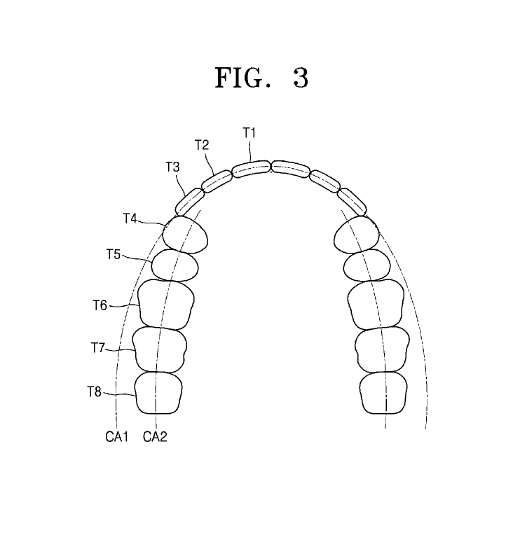

FIG. 2 is a side view of a virtual face for describing a process for generating the virtually aligned teeth information by the virtual alignment program 67 of the 3D teeth alignment image display service providing system of FIG. 1, and FIG. 3 is a bottom view of virtual teeth for describing the process for generating the virtually aligned teeth information by the virtual alignment program 67 of the 3D teeth alignment image display service providing system of FIG. 1.

Initially, as illustrated in FIG. 2, the virtual alignment program 67 may be a program for virtually moving anterior teeth in the target teeth information in such a manner that a front line FL of the anterior teeth meets an aesthetic guide line KL corresponding to a virtual line for connecting a first skin point n′ located in front of a nasion n from which nasal bones start based on a profile skull line of a person, to a second skin point p′ located in front of a pogonion p.

Then, as illustrated in FIG. 3, the virtual alignment program 67 may move the other teeth onto virtual and ideal correction arcs CA1 and CA2 based on the moved anterior teeth, thereby virtually aligning the target teeth information into the virtually aligned teeth information.

Subsequently, using the virtual teeth information, the virtual alignment program 67 may also generate virtual face contour information, virtual mouth shape information, virtual voice information, or facial expression information implementable using the aligned teeth by the orthodontic patient.

That is, the virtually aligned teeth information generated due to the above-described virtual process may include at least one of virtually corrected teeth information of the orthodontic patient, virtually corrected face contour information of the orthodontic patient, virtually corrected mouth shape information of the orthodontic patient, virtually corrected voice information of the orthodontic patient, virtually corrected facial expression information of the orthodontic patient, and combinations thereof.

The comparison program 68 may be a program for receiving a completely aligned teeth information on the completely aligned teeth of the orthodontic patient from the orthodontist terminal 20 or the orthodontic patient terminal 10, transforming an actually aligned teeth information to the three-view or four-view image information outputtable by the display 3 such that the actually aligned teeth information is projectable onto the three-sided or four-sided projection plate 2 to be compared with the virtually aligned teeth information, and transmitting the three-view or four-view image information to the orthodontist terminal 20. For example, the orthodontist may overlap and display the virtually aligned teeth information and the actually aligned teeth information of the orthodontic patient using the 3D holographic device 1, numerically display virtually and actually aligned positions, or display a rate of matches therebetween in percentage, and thus confidence and satisfaction of the orthodontic patient may be further increased. Furthermore, if the above data is provided to other orthodontic patients, an effect of advertisement or promotion may be achieved.

Herein, the above-described programs may be executive programs downloaded or installed in the orthodontic patient terminal 10, the orthodontist terminal 20, the orthodontic appliance manufacturer device 30, or the operator device 100, and may operate in association with user applications.

However, the above-described programs are not limited to executive programs or association with smartphone applications, and may operate in association with various types of devices.

Accordingly, since the orthodontist, e.g., a dentist, may accurately and realistically display various types of virtual teeth alignment information predicted after orthodontic treatment, to a patient, i.e., the orthodontic patient, as a 3D hologram using the 3D holographic device 1, reliability of the orthodontic patient in orthodontic treatment may be increased and thus confidence and satisfaction in orthodontic treatment may also be increased. Therefore, advantages in business, advertisement, or promotion may be achieved and thus differentiated and high-value-added services may be provided. Furthermore, after orthodontic treatment, other orthodontic patients may predict accuracy of orthodontic treatment by comparing actual teeth information to predicted virtual teeth information.

As illustrated in FIG. 1, the database DB may include a membership information database 81 for storing the membership information, a log-in information database 82 for storing the log-in information, a target teeth information database 83 for storing the target teeth information, a three-view or four-view image information database 84 for storing the three-view or four-view image information, a virtually aligned teeth information database 85 for storing the virtually aligned teeth information, an actually aligned teeth information database 86 for storing the actually aligned teeth information, and other information databases 87 for storing other information.

Accordingly, the server computer 60 may provide 3D teeth alignment image display services for receiving the target teeth information of the orthodontic patient from the orthodontist terminal 20 or the orthodontic patient terminal 10 using the target teeth information input program 64, virtually aligning the target teeth information into the virtually aligned teeth information using the virtual alignment program 67, transforming the virtually aligned teeth information that is obtained based on the target teeth information, into the three-view or four-view image information outputtable by the display 3 such that the virtually aligned teeth information is projectable onto the three-sided or four-sided projection plate 2, using the three-view or four-view image information transformation program 65, transmitting the three-view or four-view image information to the orthodontist terminal 20 using the three-view or four-view image information transmission program 66, and receiving a completely aligned teeth information on the completely aligned teeth of the orthodontic patient from the orthodontist terminal 20 or the orthodontic patient terminal 10, transforming an actually aligned teeth information into the three-view or four-view image information outputtable by the display 3 such that the actually aligned teeth information is projectable onto the three-sided or four-sided projection plate 2 to be compared with the virtually aligned teeth information, and transmitting the actually aligned teeth information to the orthodontist terminal 20, using the comparison program 68.

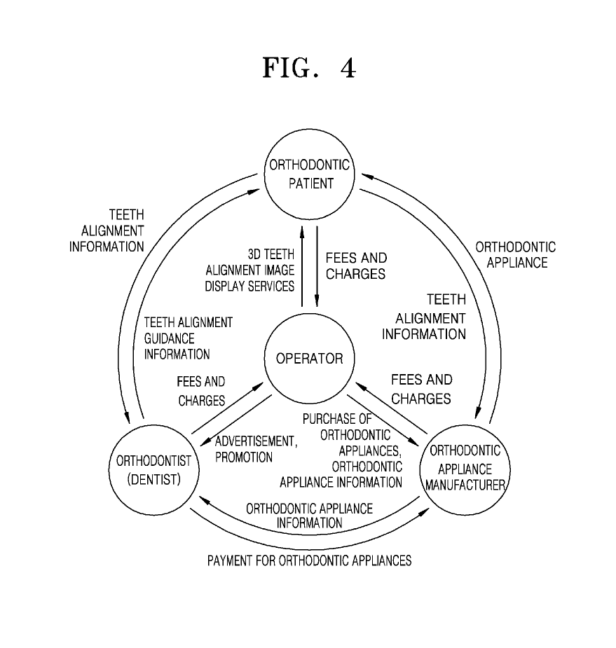

FIG. 4 is a conceptual view showing the relationship among the operator for providing 3D teeth alignment image display services of the present invention, the orthodontic patient, the orthodontist, and the orthodontic appliance manufacturer.

Therefore, as illustrated in FIG. 4, the operator for operating the server computer 60 may receive fees and charges for the 3D teeth alignment image display services from the orthodontic patient, the orthodontist, and the orthodontic appliance manufacturer. The orthodontic patient, the orthodontist, and the orthodontic appliance manufacturer may receive the high-quality 3D teeth alignment image display services, teeth alignment information, orthodontic appliance information, or the like. As such, confidence and satisfaction may be provided to orthodontic patients, advantages in business, advertisement, or promotion may be achieved, and thus a business model which is advantageous and useful to all may be provided.

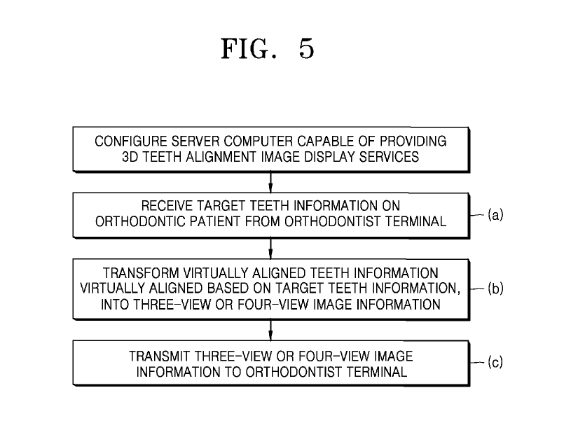

FIG. 5 is a flowchart of a method of providing 3D teeth alignment image display services, according to some embodiments of the present invention.

As illustrated in FIGS. 1 to 5, the 3D teeth alignment image display service providing method according to some embodiments of the present invention may sequentially include configuring the server computer 60 capable of providing the 3D teeth alignment image display services, (a) receiving a target teeth information on the orthodontic patient from the orthodontist terminal 20 or the orthodontic patient terminal 10 using the target teeth information input program 64, (b) transforming a virtually aligned teeth information that is obtained based on the target teeth information, to a three-view or four-view image information outputtable by the display 3 such that the virtually aligned teeth information is projectable onto the three-sided or four-sided projection plate 2, using the three-view or four-view image information transformation program 65, and (c) transmitting the three-view or four-view image information to the orthodontist terminal 20 using the three-view or four-view image information transmission program 66.

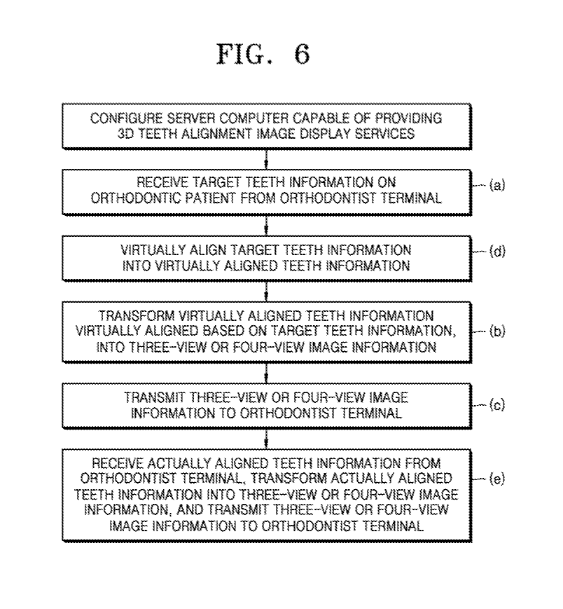

FIG. 6 is a flowchart of a method of providing 3D teeth alignment image display services, according to other embodiments of the present invention.

As illustrated in FIGS. 1 to 6, the 3D teeth alignment image display service providing method according to other embodiments of the present invention may sequentially include configuring the server computer 60 capable of providing the 3D teeth alignment image display services, (a) receiving a target teeth information on the orthodontic patient from the orthodontist terminal 20 or the orthodontic patient terminal 10 using the target teeth information input program 64, (d) virtually aligning the target teeth information into virtually aligned teeth information using the virtual alignment program 67, (b) transforming the virtually aligned teeth information virtually aligned based on the target teeth information, to a three-view or four-view image information outputtable by the display 3 such that the virtually aligned teeth information is projectable onto the three-sided or four-sided projection plate 2, using the three-view or four-view image information transformation program 65, (c) transmitting the three-view or four-view image information to the orthodontist terminal 20 using the three-view or four-view image information transmission program 66, and (e) receiving a completely aligned teeth information on completely aligned teeth of the orthodontic patient from the orthodontist terminal 20 or the orthodontic patient terminal 10, transforming an actually aligned teeth information to a three-view or four-view image information outputtable by the display 3 such that the actually aligned teeth information is projectable onto the three-sided or four-sided projection plate 2 to be compared with the virtually aligned teeth information, and transmitting the three-view or four-view image information to the orthodontist terminal 20, using the comparison program 68.

Accordingly, since the orthodontist may provide a 3D teeth alignment image to the orthodontic patient using the 3D holographic device 1, reliability of the orthodontic patient in orthodontic treatment may be increased, confidence and satisfaction in orthodontic treatment may also be increased, and thus the orthodontic patient may easily decide to receive orthodontic treatment. Therefore, advantages in business, advertisement, or promotion may be achieved and thus differentiated and high-value-added services may be provided. Furthermore, after orthodontic treatment, other orthodontic patients may predict accuracy of orthodontic treatment by comparing actual teeth information to predicted virtual teeth information. Therefore, more advantages in business, advertisement, or promotion may be achieved.

As described above, according to some embodiments of the present invention, since an orthodontist may provide a 3D teeth alignment image using a 3D holographic device, reliability of an orthodontic patient in orthodontic treatment may be increased and thus confidence and satisfaction in orthodontic treatment may also be increased. Therefore, advantages in business, advertisement, or promotion may be achieved and thus differentiated and high-value-added services may be provided. Furthermore, after orthodontic treatment, other orthodontic patients may predict accuracy of orthodontic treatment by comparing actual teeth information to predicted virtual teeth information. However, the scope of the present invention are not limited to the above-described effects.

The present invention can also be embodied as computer-readable code on a computer-readable medium.

The computer-readable medium is any data storage device that can store data which can be thereafter read by a computer system.

Examples of the computer-readable medium include not only the above-described server computer 60 but also read-only memory (ROM), random-access memory (RAM), CD-ROMs, magnetic tapes, floppy disks, magneto-optical disks, optical data storage devices, flash memory, and carrier waves (e.g., transmission through the Internet).

The computer-readable medium can also be distributed over network coupled computer systems so that the computer-readable code is stored and executed in a distributed fashion.

While the present invention has been particularly shown and described with reference to embodiments thereof, it will be understood by one of ordinary skill in the art that various changes in form and details may be made therein without departing from the spirit and scope of the present invention as defined by the following claims.

What is claimed is:

1. A method of providing 3D teeth alignment image display services using a computer system comprising at least one orthodontist terminal and a server computer connected to a network, the method comprising:
receiving a target teeth information on an orthodontic patient from the orthodontist terminal or an orthodontic patient terminal such that three or four images from a display are respectively projected onto a substantially pyramidal three-sided or four-sided projection plate and thus the orthodontic patient checks a 3D hologram of his/her virtually aligned teeth,
transforming a virtually aligned teeth information that is obtained based on the target teeth information, to three-view or four-view image information outputtable by the display such that the virtually aligned teeth information is projectable onto the three-sided or four-sided projection plate, and
transmitting the three-view or four-view image information to the orthodontist terminal.

2. A method of providing 3D teeth alignment image display services using a computer system comprising at least one orthodontist terminal and a server computer connected to a network, the server computer comprising:
a target teeth information input program for receiving a target teeth information on an orthodontic patient from the orthodontist terminal or an orthodontic patient terminal such that three or four images from a display are respectively projected onto a pyramidal three-sided or four-sided projection plate and thus the orthodontic patient checks a 3D hologram of his/her virtually aligned teeth;
a three-view or four-view image information transformation program for transforming a virtually aligned teeth information that is obtained based on the target teeth information, to three-view or four-view image information outputtable by the display such that the virtually aligned teeth information is projectable onto the three-sided or four-sided projection plate;
a three-view or four-view image information transmission program for transmitting the three-view or four-view image information to the orthodontist terminal;
a target teeth information database for storing the target teeth information; and
a three-view or four-view image information database for storing the three-view or four-view image information, and
the method comprising:
(a) receiving the target teeth information on the orthodontic patient from the orthodontist terminal or the orthodontic patient terminal using the target teeth information input program;
(b) transforming the virtually aligned teeth information that is obtained based on the target teeth information, to the three-view or four-view image information outputtable by the display such that the virtually aligned teeth information is projectable onto the three-sided or four-sided projection plate, using the three-view or four-view image information transformation program; and
(c) transmitting the three-view or four-view image information to the orthodontist terminal using the three-view or four-view image information transmission program.

3. The method of claim 2, wherein the target teeth information comprises at least one of:
a teeth photo information obtained by photographing teeth or dental impressions of the orthodontic patient;
a 3D-scanned teeth information obtained by 3D-scanning the teeth or the dental impressions of the orthodontic patient;
a face photo information obtained by photographing a face of the orthodontic patient;
a 3D-scanned face information obtained by 3D-scanning the face of orthodontic patient;
a current mouth shape information of the orthodontic patient;
a current voice information of the orthodontic patient;
a current facial expression information of the orthodontic patient; and
combinations thereof.

4. The method of claim 2, wherein the three-view or four-view image information comprises at least one of a front view image information, a left side view image information, a right side view image information, and a rear view image information and combinations thereof, indicating virtually aligned teeth or face contours of the orthodontic patient, and
wherein the front view image information, the left side view image information, the right side view image information, and the rear view image information are information on images which are radially provided in three or four equiangular directions about a point that corresponds to an apex of the three-sided or four-sided projection plate and thus are respectively projectable onto each side of the three-sided or four-sided projection plate.

5. The method of claim 2, wherein the server computer further comprises:
a virtual alignment program for virtually aligning the target teeth information into the virtually aligned teeth information; and
a virtually aligned teeth information database for storing the virtually aligned teeth information, and
wherein the method further comprises:
(d) virtually aligning the target teeth information into the virtually aligned teeth information using the virtual alignment program, after step (a).

6. The method of claim 5, wherein the virtual alignment program is a program for virtually aligning the target teeth information into the virtually aligned teeth information by virtually moving anterior teeth in the target teeth information such that a front line of the anterior teeth meets an aesthetic guide line corresponding to a virtual line for connecting a first skin point located in front of a nasion from which nasal bones start based on a profile skull line of a person, to a second skin point located in front of a pogonion, and then moving other teeth onto virtual and ideal correction arcs based on the moved anterior teeth.

7. The method of claim 2, wherein the virtually aligned teeth information comprises at least one of:
a virtually corrected teeth information of the orthodontic patient;
a virtually corrected face contour information of the orthodontic patient;
a virtually corrected mouth shape information of the orthodontic patient;
a virtually corrected voice information of the orthodontic patient;
a virtually corrected facial expression information of the orthodontic patient; and
combinations thereof.

8. The method of claim 2, wherein the server computer further comprises:
a comparison program for receiving a completely aligned teeth information on completely aligned teeth of the orthodontic patient from the orthodontist terminal or the orthodontic patient terminal, transforming an actually aligned teeth information to three-view or four-view image information outputtable by the display such that the actually aligned teeth information is projectable onto the three-sided or four-sided projection plate to be compared with the virtually aligned teeth information, and transmitting the three-view or four-view image information to the orthodontist terminal; and
an actually aligned teeth information database for storing the actually aligned teeth information, and
wherein the method further comprises:
(e) receiving the completely aligned teeth information on the completely aligned teeth of the orthodontic patient from the orthodontist terminal or the orthodontic patient terminal, transforming the actually aligned teeth information to the three-view or four-view image information outputtable by the display such that the actually aligned teeth information is projectable onto the three-sided or four-sided projection plate to be compared with the virtually aligned teeth information, and transmitting the three-view or four-view image information to the orthodontist terminal, using the comparison program, after step (c).

9. A computer system for providing 3D teeth alignment image display services using a computer system comprising at least one orthodontist terminal and a server computer connected to a network, the server computer comprising:
a target teeth information input program for receiving a target teeth information on an orthodontic patient from the orthodontist terminal or an orthodontic patient terminal such that three or four images from a display are respectively projected onto a substantially pyramidal three-sided or four-sided projection plate and thus the orthodontic patient checks a 3D hologram of his/her virtually aligned teeth;
a three-view or four-view image information transformation program for transforming a virtually aligned teeth information that is obtained based on the target teeth information, to three-view or four-view image information outputtable by the display such that the virtually aligned teeth information is projectable onto the three-sided or four-sided projection plate;
a three-view or four-view image information transmission program for transmitting the three-view or four-view image information to the orthodontist terminal;
a target teeth information database for storing the target teeth information;
a three-view or four-view image information database for storing the three-view or four-view image information; and
a controller programmed to receive the target teeth information on the orthodontic patient from the orthodontist terminal or the orthodontic patient terminal using the target teeth information input program, to transform the virtually aligned teeth information that is obtained based on the target teeth information, to the three-view or four-view image information outputtable by the display such that the virtually aligned teeth information is projectable onto the three-sided or four-sided projection plate, using the three-view or four-view image information transformation program, and to transmit the three-view or four-view image information to the orthodontist terminal using the three-view or four-view image information transmission program.

10. A computer-readable medium on which is recorded a method of providing 3D teeth alignment image display services using a computer system comprising at least one orthodontist terminal and a server computer connected to a network, the server computer comprising:
a target teeth information input program for receiving a target teeth information on an orthodontic patient from the orthodontist terminal or an orthodontic patient terminal such that three or four images from a display are respectively projected onto a pyramidal three-sided or four-sided projection plate and thus the orthodontic patient checks a 3D hologram of his/her virtually aligned teeth;
a three-view or four-view image information transformation program for transforming a virtually aligned teeth information that is obtained based on the target teeth information, to three-view or four-view image information outputtable by the display such that the virtually aligned teeth information is projectable onto the three-sided or four-sided projection plate;
a three-view or four-view image information transmission program for transmitting the three-view or four-view image information to the orthodontist terminal;
a target teeth information database for storing the target teeth information; and
a three-view or four-view image information database for storing the three-view or four-view image information, and
the method comprising:
(a) receiving the target teeth information on the orthodontic patient from the orthodontist terminal or the orthodontic patient terminal using the target teeth information input program;
(b) transforming the virtually aligned teeth information that is obtained based on the target teeth information, to the three-view or four-view image information outputtable by the display such that the virtually aligned teeth information is projectable onto the three-sided or four-sided projection plate, using the three-view or four-view image information transformation program; and
(c) transmitting the three-view or four-view image information to the orthodontist terminal using the three-view or four-view image information transmission program.
